(19) United States
(12) Patent Application Publication
(10) Pub. No.: US 2010/0040669 A1
(43) Pub. Date: Feb. 18, 2010

(54) Non-Invasive Ocular Delivery of Rapamycin

(76) Inventor: John W. Higuchi, Park City, UT (US)

Correspondence Address: THORPE NORTH & WESTERN, LLP., P.O. Box 1219, SANDY, UT, 84091-1219, US

(21) Appl. No.: 12/540,271

(22) Filed: Aug. 12, 2009

Related U.S. Application Data
(60) Provisional application No. 61/088,245, filed on Aug. 12, 2008.

(51) Int. Cl. A61F 2/00; A61K 31/436

(52) U.S. Cl. 424/427; 514/291

(57) ABSTRACT

Methods and systems for preventing or treating various ocular conditions are disclosed and described. In one aspect, for example, a method for noninvasively delivering a water insoluble macrolide into an eye of a subject for treatment of an ocular condition is provided. Such a method may include administering non-invasively a water soluble form of the macrolide directly into an eye of a subject having or at risk for having the ocular condition, wherein the water soluble form is converted into the water insoluble macrolide in the eye in order to treat the ocular condition.

### PRIORITY DATA

This application claims the benefit of U.S. Provisional Patent Application Ser. No. 61/088,245, filed on Aug. 12, 2008, which is incorporated herein by reference.

### FIELD OF THE INVENTION

The present invention relates to systems and methods for treating ocular conditions. Accordingly, the present invention involves the fields of chemistry, pharmaceutical sciences, and medicine, particularly opthalmology.

### BACKGROUND OF THE INVENTION

Certain conditions of the eye have proven challenging to treat. In many cases posterior and intermediate eye conditions may require ocular drug delivery to prevent blindness. Examples of such conditions include uveitis, age-related macular degeneration, viral retinitis, and diabetic retinopathy, among others. The reported incidence of posterior uveitis, for example, is more than 100,000 people in the United States. If left untreated, uveitis leads to blindness, being responsible for about 10 percent of all visual impairment in the U.S. and is the third leading cause of blindness worldwide.

Treatments of intermediate and posterior eye conditions are complicated due to inaccessibility of the posterior eye to topically applied medications. Current therapy for many intermediate and posterior eye conditions requires repeated periocular injections and/or high-dose systemic therapy with corticosteroids. Systemic drug administration is usually not preferred because the blood/retinal barrier impedes the passage of most drugs from the systemically circulating blood to the interior of the eye. Therefore large systemic doses are needed to treat many intermediate and posterior eye conditions, which often result in systemic toxicities including immunosuppression, adrenal suppression, ulcerogenesis, fluid and electrolyte imbalances, fat redistribution and psychological disorders. For example, corticosteroids such as triamcinolone acetonide and dexamethasone have been used as standard treatments for controlling intraocular inflammation. However, such drugs have side effects that may lead to increased intraocular pressure, thus increasing the risk of glaucoma and cataracts.

As such, systems and methods which are capable of treating or preventing ocular conditions continue to be sought.

### SUMMARY OF THE INVENTION

Accordingly, the present invention provides systems and methods of treating various ocular conditions. In one aspect, for example, a method for noninvasively delivering a water insoluble macrolide into an eye of a subject for treatment of an ocular condition is provided. Such a method may include administering non-invasively a water soluble form of the macrolide directly into an eye of a subject having or at risk for having the ocular condition, wherein the water soluble form is converted into the water insoluble macrolide in the eye in order to treat the ocular condition.

Though various macrolides are contemplated, any such active agent known may be administered according to the methods disclosed herein. In one aspect, for example, the macrolide can include SDZ-RAD, tacrolimus, everolimus, pimecrolimus, CCI-779, AP23841, ABT-578, cyclophilins, FK506-binding proteins (FKBPs), TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, ascomycin, and pharmaceutically acceptable prodrugs, analogs, salts, and esters thereof. In another aspect, the macrolide can be a water soluble rapamycin agent. In yet another aspect, the macrolide can be a prodrug of rapamycin. In a further aspect, the macrolide can include a member selected from the group consisting of ascomycin, and pharmaceutically acceptable prodrugs, analogs, salts, and esters thereof.

Aspects of the present invention may be used to treat a variety of ocular conditions. Any ocular condition that may be treated using a macrolide is considered to be within the present scope. Non-limiting examples may include wherein the ocular condition is a member selected from the group consisting of chronic macular edema, diabetic macular edema, cystoid macular edema, macular edema, age related macular degeneration, diabetic retinopathy, urticaria, allergic conjunctivitis, vernal conjunctivitis, inflammation of the eye, allergic responses in the eye, uveitis, Behcet's disease, pars planitis, idiopathic uveitis, ocular sarcoid, sympathetic ophthalmia, idiopathic vitritis, vitritis, uveitis resulting from trauma, iritis, iridocyclitis, scleritis, episcleritis, choroiditis, optic neuritis, Mooren's ulcer, ulcerative keratitis associated with rheumatoid arthritis, anterior uveitis, Thygeson's punctate keratitis, retinitis pigmentosa, diabetic retinopathy, chronic glaucoma, retinal detachment, sickle cell retinopathy, senile macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, retinal vein occlusion, proliferative vitreoretinopathy, angioid streak, retinal artery occlusion, neovascularization due to ocular injury, infections of the eye by infective agents including viruses, fungi, bacteria, and combinations thereof. In one specific aspect, the ocular condition may include macular edema and/or diabetic macular edema. In another specific aspect, the ocular condition may include age related macular degeneration.

In another aspect of the present invention, the macrolide may be co-administered with a vasoconstrictor to further enhance the treatment outcome of the ocular condition. Various vasoconstrictors may be co-administered with the steroid-sparing immunosuppressive agents according to aspects of the present invention. For example, in one specific aspect, the vasoconstrictor may be an α-agonist. Examples of α-agonists may include, without limitation, naphazoline, tetrahydrozoline, oxymetazoline, and combinations thereof. In another specific aspect, the vasoconstrictor may be a sympathomimetic amine. Non-limiting examples of sympathomimetic amines may include phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, albuterol, amphetamine, methamphetamine, benzphetamine, ephedrine, phenylpropanolamine, methentermine, phentermine, fenfluramine, propylhexedrine, diethylpropion, phenmetrazine, phendimetrazine, and combinations thereof. Finally, in addition to vasoconstrictors, various other compounds may be co-administered with the macrolide, including various permeation enhancers.

The macrolides of the present invention may be administered into the eye by a variety of mechanisms. In one example, the macrolide may be delivered via an active delivery mechanism such as iontophoresis. In another example, the agent may be passively administered via topical application to the eye.

The present invention also provides systems for treating or preventing an ocular condition as has been described. In one aspect, for example, a system may include an ocular delivery drug device having at least one drug reservoir and a macrolide contained within the drug reservoir.

### BRIEF DESCRIPTION OF THE DRAWINGS

FIG. 1 is a front view of an iontophoretic device in accordance with an aspect of the present invention.

### DETAILED DESCRIPTION OF THE INVENTION

Before the present systems and methods for the treatment or prevention of eye conditions are disclosed and described, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “a polymer” includes reference to one or more of such polymers, and “the excipient” includes reference to one or more of such excipients.

### DEFINITIONS

In describing and claiming the present invention, the following terminology will be used in accordance with the definitions set forth below.

As used herein, “formulation” and “composition” may be used interchangeably herein, and refer to a combination of two or more elements, or substances. In some embodiments a composition may include an active agent and a carrier.

As used herein, “drug,” “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. These terms of art are well-known in the pharmaceutical, and medicinal arts.

As used herein, the term “macrolide” refers to a drug having a macrolide ring. Non-limiting examples of macrolides include SDZ-RAD, tacrolimus, everolimus, pimecrolimus, CCI-779, AP23841, ABT-578, cyclophilins, FK506-binding proteins (FKBPs), TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, ascomycin, and pharmaceutically acceptable prodrugs, analogs, salts, and esters thereof.

As used herein, the term “rapamycin agent” is used to refer to drugs that are prodrugs, analogs, salts, and esters of rapamycin, excluding rapamycin itself.

As used herein, “derivative,” when used in association with a drug, a biological or a chemical compound, refers to the drug, the biological or the chemical compound and any analog, homolog, prodrug, isomer, enantiomer, acid addition salt, free base, metabolite, or combination thereof, of that drug, biological or a chemical compound.

As used herein “prodrug” refers to a molecule that will convert under physiological conditions into a drug (its commonly known pharmacologically active form). It should be understood that a prodrug can also be pharmacologically active.

The term “vasoconstrictor” refers to any compound capable of decreasing the diameter of a vascular vessel by any of a variety of pharmacological mechanisms.

As used herein, “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference. In addition, “therapies” are not necessarily stand alone therapies but can be combinatory therapies or adjunctive therapies for the prevention and treatment of a disease.

As used herein, “carrier” or “inert carrier” refers to a substance with which a drug may be combined to achieve a specific dosage formulation for delivery to a subject. In the some aspects of the present invention, the carriers used may or may not enhance drug delivery. As a general principle, carriers do not react with the drug in a manner which substantially degrades or otherwise adversely affects the drug, except that carriers may react with a drug to prevent it from exerting a therapeutic effect until the drug is released from the carrier. Further, the carrier, or at least a portion thereof must be suitable for administration into a subject along with the drug.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “eye” and “ocular” refer to the peripheral visual organ of a subject.

As used herein, “administration,” and “administering” refer to the manner in which an active agent, drug, or composition containing such, is presented to a subject. As discussed herein, the present invention is primarily concerned with mechanisms for ocular delivery of an active agent or composition.

As used herein, “noninvasive” refers to a form of administration that does not rupture or puncture a biological membrane or structure with a mechanical means across which a drug or compound of interest is being delivered. A number of noninvasive delivery mechanisms are well recognized in the transdermal arts, such as, for example, patches and topical formulations. Many of such formulations can employ a chemical penetration enhancer in order to facilitate non-invasive delivery of the active agent. Additionally, other systems or devices that utilize a non-chemical mechanism for enhancing drug penetration, such as iontophoretic devices are also known. In addition, “minimally invasive” refers to a form of administration that does minimal damage to a biological membrane or structure as compared to intravitreal injection or surgical procedure, when using a mechanical or chemical mechanism across which a drug or compound of interest is delivered. For example, and without limitation, minimally invasive ocular delivery includes periocular injections using a small needle. Such injections can include, for example, subconjunctival injection, sub-Tenon's injection, retrobulbar injection, and peribulbar injection. All such injections are much less invasive than intravitreal injection and surgical device implantation.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### THE INVENTION

The present invention provides methods, systems, and devices for the treatment or prevention of various ocular conditions. As has been described, prior methods for treating many ocular conditions include systemically administering a drug such as a corticosteroid to a subject. Such systemic administration has proven difficult for a number of reasons, including side effects from the large doses of drug required to treat the eye due to the blood/retinal barrier that impedes the passage of most systemically circulating drugs into the interior of the eye. Also, high doses of systemically administered corticosteroids often increase the incidence of side effects that may include immunosuppression, adrenal suppression, ulcerogenesis, fluid and electrolyte imbalances, fat redistribution, psychological disorders, etc.

The inventors have thus discovered that various ocular conditions may be treated by administering a macrolide directly into the eye. Many of these compounds have steroid sparing effects, and thus have the potential of treating ocular conditions without the side effects associated with steroids. Because a macrolide delivered directly to the eye can be administered in lower amounts as compared to systemic therapies, systemic exposure to the macrolide is decreased. Accordingly, side effects are minimized as a result of reduced systemic exposure. These smaller, more tolerable doses of a macrolide can be used as the sole treatment for the ocular condition, or the macrolide can be used in combination with another ocular or systemic drug therapy to treat the condition. Such a combination therapy with a systemic drug can allow lower doses of the systemic drug to be administered, thus lowering the incidence of side effects.

One difficulty that can be associated with the non-invasive delivery of some macrolides into the eye is the low water solubility of many such compounds. Drugs having low water solubility can be difficult to administer non-invasively. As such, it can be difficult to achieve therapeutic levels of these macrolides through such non-invasive means. As an example, rapamycin has a low water solubility on the order of about 2.8 μM, and as such, cannot generally be non-invasively administered in a therapeutically effective amount through ocular tissues. Additionally, rapamycin degrades quickly in physiological environments, and thus what little drug can be delivered into the eye will quickly be degraded before reaching therapeutic levels. The inventors have discovered that such difficulties can be overcome by locally administering water soluble forms of rapamycin and other macrolides into the eye. Additionally, water soluble macrolides that convert to an insoluble moiety will cause the formation of an insoluble drug depot inside the eye, thus allowing sustained release of the compound over time.

Numerous macrolides are contemplated for treating a variety of ocular conditions. Macrolides are generally a group of drugs whose activity stems from the presence of a macrolide ring, to which one or more deoxy sugars, usually cladinose and desosamine, may be attached. Such macrocyclic rings are often 14, 15, or 16-membered. In one aspect, for example, the macrolide can include SDZ-RAD, tacrolimus, everolimus, pimecrolimus, CCl-779, AP23841, ABT-578, cyclophilins, FK506-binding proteins (FKBPs), TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, ascomycin, and pharmaceutically acceptable prodrugs, analogs, salts, and esters thereof. In another aspect, the macrolide can be a water soluble rapamycin agent. In yet another aspect, the macrolide can be a prodrug of rapamycin. In a further aspect, the macrolide can include a member selected from the group consisting of ascomycin, and pharmaceutically acceptable prodrugs, analogs, salts, and esters thereof.

As has been described, of particular interest are water soluble macrolide drugs. In many cases, water soluble macrolides are more easily delivered to the eye by noninvasive means as compared to water insoluble macrolides. As has been described, such non invasive methods exclude “minimally invasive” transscleral methods such as microneedles, subconjunctival or periocular injections, etc. However, non invasive methods may include administration techniques such as, without limitation, topical eyedrops, ocular minitabs or biodegradable polymer inserts on the eye's surface, ocular iontophoresis, passive diffusion following poration methods, contact lens shaped applicators (corneal or scleral), etc.

The present invention thus provides methods for treating an ocular condition by administering compositions containing macrolide orthophosphates and associated prodrugs. As has been described above, macrolides encompass numerous drug compounds. Two exemplary categories include, without limitation, rapamycin (Structure I) and ascomycin (Structure II). Rapamycin, however is a rather water insoluble drug, and therefore difficult to administer by noninvasive means. In one aspect, therefore, water soluble rapamycin prodrugs can be readily delivered by noninvasive means. Once in the eye, these prodrugs are converted to the drug by enzyme activity of phosphatase enzymes present in tissues, thus creating pharmacologically effective doses of the drugs in the tissues. It should be noted that this situation also applies to other water-insoluble macrolide compounds.

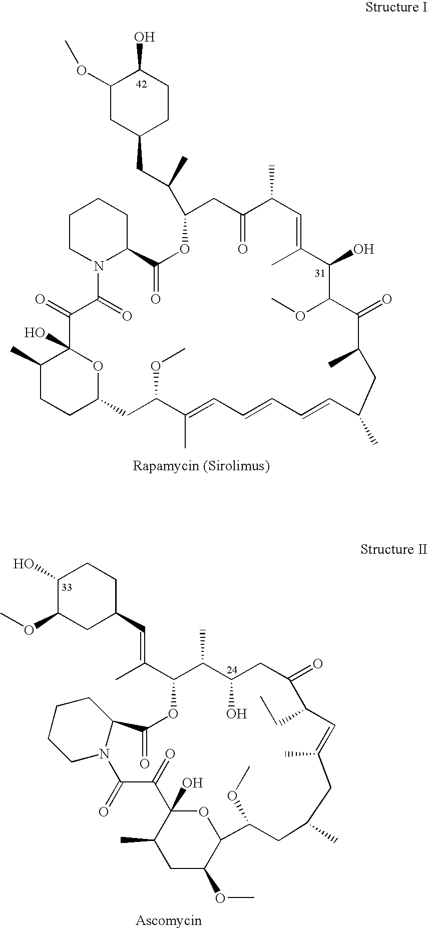

In one aspect, therefore, a method for treatment may include the non invasive, controlled release or sustained release treatment of an eye disease that delivers a therapeutically relevant dosing of the drug to the retina/choroid, back of the eye, and/or posterior pole of the eye, using a water soluble formulation of rapamycin. Various water soluble formulations are contemplated, non-limiting examples of which are described below. The formulation is applied topically or non-invasively to the eyeball's surface whether the drug reservoir is in direct contact with the conjunctiva or cornea surface areas.

Examples of various rapamycin agent compounds, formulations, etc., can be found in US patents U.S. Pat. No. 5,389,639, U.S. Pat. No. 6,432,973, U.S. Pat. No. 4,650,803, U.S. Pat. No. 5,530,120, U.S. Pat. No. 5,391,730, U.S. Pat. No. 5,080,899, U.S. Pat. No. 5,023,263, U.S. Pat. No. 7,160,867, U.S. Pat. No. 5,091,389, U.S. Pat. No. 5,665,772, EP 0227355, EP 0429436, and WO/2000058318, each of which is incorporated herein by reference.

As an example, two potential water soluble prodrug categories are shown in structure III and structure IV, which have high water solubility at pH 7.0, are ionizable, and can be formulated in aqueous media. Such prodrugs provide new formulations that are deliverable through non-invasive technologies such as iontophoresis or topical applications.

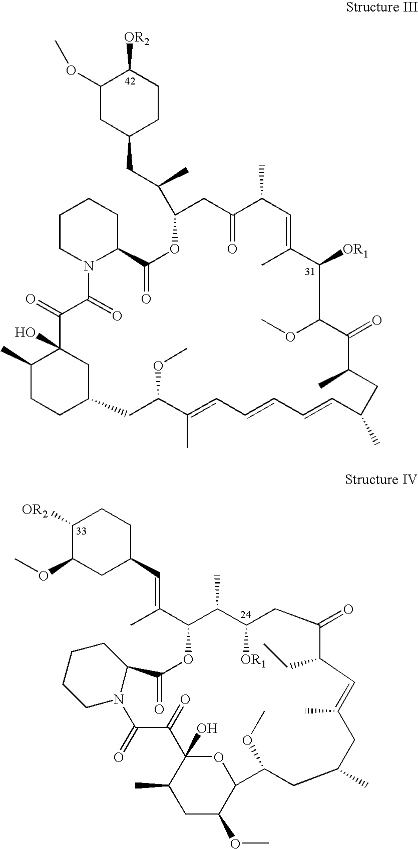

where R1 and R2 are independently:

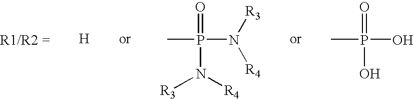

R1 and/or R2 are a diamidophosphoryl or a phosphate group where R3 and R4 are alkyl or aralkyl groups, or when R3 and R4 are linked directly or via another heteroatom and together with the nitrogen atom to form a saturated heterocyclic ring (e.g. morpholin, pyrrolidine etc.).

The compounds of formula corresponding to Structure III and Structure IV are synthesized starting from macrolides (for example, structure I and structure II above) by the reaction of a diamidophosphoryl halide. The resulting diamidoorthophosphate of the macrolide is then treated with acid to hydrolyze the amide functions and obtain the macrolide orthophosphates.

Thus the formulations of macrolides, including compounds derived from the structures shown in structure III and structure IV, can be delivered noninvasively using an active or a passive delivery system. Further examples of active and passive delivery systems can be found in U.S. patent application Ser. Nos. 11/999,266, filed on Dec. 3, 2007, 11/867,503 filed on Oct. 4, 2007, 11/414,134 filed on Apr. 27, 2006, 11/414,137 filed on Apr. 27, 2006, 11/238,144 filed on Sep. 27, 2005, and 11/238,104 filed on Sep. 27, 2005, each of which is incorporated herein by reference.

Various compounds may be co-administered with the macrolide agents as described herein. In one aspect, for example, a vasoconstrictor may be co-administered with the macrolide agent. One benefit of such a formulation is the prodrug would be subject to reduced blood flow in the eye tissue, and thus experience fewer clearance effects. This can allow more time for the respective phosphatase or esterase enzymes present in the eye tissue to convert the water soluble prodrug into an insoluble form. Additional details regarding vasoconstrictor administration can be found in U.S. patent application Ser. No. 10/269,911, filed on Oct. 11, 2002, which is incorporated herein by reference.

Any vasoconstrictor that can benefit the administration of a macrolide in the eye should be considered to be within the present scope. In one aspect, for example, the vasoconstrictor can be an α-agonist. Examples of α-agonists can include naphazoline, tetrahydrozoline, and oxymetazoline. In another aspect, the vasoconstrictor can be a sympathomimetic amine. Non-limiting examples of sympathomimetic amines include phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, albuterol, amphetamine, methamphetamine, benzphetamine, ephedrine, phenylpropanolamine, methentermine, phentermine, fenfluramine, propylhexedrine, diethylpropion, phenmetrazine, phendimetrazine, and combinations thereof. In one specific aspect, the vasoconstrictor is oxymetazoline.

Additionally, a wide variety of additional drugs may be co-administered to the eye of a subject with the macrolide. Of course, selection of a specific additional drug and the specific form of that drug will depend on a variety of considerations, such as the specific ocular condition to be treated or prevented, the specific carrier to be used, the duration of the desired treatment, and any overriding health considerations of the subject, such as allergies to certain medications. However, as a general matter, a number of specific additional drugs are known as useful in treating one or more of the ocular conditions recited herein, including without limitation, steroids, steroid derivatives such as aminosteroids, antibacterials, antivirals, antifungals, antiprotozoals, antimetabolites, VEGF inhibitors, ICAM inhibitors, antibodies, protein kinase C inhibitors, chemotherapeutic agents, neuroprotective agents, nucleic acid derivatives, aptamers, proteins, enzymes, peptides, polypeptides. More specific examples include without limitation dexamethasone phosphate, triamcinolone derivatives such as triamcinolone acetonide, triamcinolone acetonide phosphate, squalamine, amikacin, oligonucleotides, Fab peptides, PEG-oligonucleotides, salicylate, tropicamide, methotrexate, 5-fluorouracil, and diclofenac. Finally, in addition to vasoconstrictors, various other compounds may be co-administered with the macrolide, including various permeation enhancers that are well known in the art.

Any condition of the eye that is treatable with a macrolide is considered to be within the scope of the present invention. Benefit can be derived from such a treatment for those conditions including diseases whereby a macrolide is most effective when administered directly to the eye. Non-limiting examples of eye-related conditions that may be treatable through application of a macrolide according to aspects of the present invention include chronic macular edema, age related macular degeneration, diabetic retinopathy, urticaria, allergic conjunctivitis, vernal conjunctivitis, inflammation of the eye, allergic responses in the eye, uveitis, Behcet's disease, pars planitis, idiopathic uveitis, ocular sarcoid, sympathetic ophthalmia, idiopathic vitritis, vitritis, uveitis resulting from trauma, iritis, iridocyclitis, scleritis, episcleritis, choroiditis, optic neuritis, Mooren's ulcer, ulcerative keratitis associated with rheumatoid arthritis, anterior uveitis, Thygeson's punctate keratitis, retinitis pigmentosa, and other immunological reactions. Additionally, ocular conditions may include infections of the eye by infective agents such as viruses including adenovirus and cytomegalovirus, fungi including toxoplasmosis, bacteria including tuberculosis and syphilis, chlamydia, and amoeba. It should be noted that any condition that can be treated by administering a macrolide directly into the eye should be considered to be within the scope of the present invention.

There are a number of non-invasive drug delivery techniques that are suitable for delivery of a drug into a subject's eye. It should be noted that any means of administering compounds to the eye of a subject should be considered to be within the scope of the present invention. In one aspect, for example, solutions and suspensions that can be administered in the form of drops can be used. In yet another aspect, iontophoretic devices and methods may be used to non-invasively administer drugs into the eye that may be particularly successful in achieving a high degree of drug penetration with a short duration. Therefore, subject discomfort and inconvenience are minimized, as well as the risk of certain potential adverse side effects for the treatment regimen as a whole. Additionally, ocular devices can be used to passively deliver the present formulations into the eye.

Accordingly, in one aspect of the invention, non-invasive administration mechanism may be iontophoretic administration. A number of specific iontophoretic devices and configurations may be suitably used to deliver drugs into the eye, all of which are suitable for use in the present invention. Specific examples of useful iontophoretic techniques include, without limitation, alternating current (AC), direct current (DC), AC with superimposed DC offset, and electroporation. In practice, two iontophoretic electrodes are used in order to complete an electrical circuit. In traditional transscleral iontophoresis, at least one of the electrodes is considered to be an active iontophoretic electrode, while the other may be considered as a return, inactive, or indifferent electrode. The active electrode is typically placed on an eye surface. The compound of interest is transported at the active electrode across the tissue as a permeant when a current is applied to the electrodes through the tissue. Compound transport may occur as a result of a direct electrical field effect (e.g., electrophoresis), an indirect electrical field effect (e.g., electroosmosis), electrically induced pore or transport pathway formation (electroporation), or a combination of any of the foregoing. Examples of currently known iontophoretic devices and methods for ocular drug delivery may be found in U.S. Pat. Nos. 6,319,240; 6,539,251; 6,579,276; 6,697,668, and PCT Publication Nos. WO 03/030989 and WO 03/043689, each of which is incorporated herein by reference.

For optimal iontophoretic delivery of the macrolide agent into the eye, the methods and systems of the present invention can further include placing a permselective material in ion-conducting relation to the eye surface. An electric current of AC, DC, or AC with superimposed DC can be used to drive the compounds of interest through the permselective material to the eye. The permselective material hinders iontophoretic transport of a competing ion and increases the transference efficiency of the compound of interest during iontophoresis. As a result, the compounds and carriers of interest are delivered iontophoretically into the eye more efficiently than without the permselective material.

Any permselective material capable of hindering iontophoretic transport of a competing ion during iontophoretic transport of the compounds and carriers of interest may be used in conjunction with the invention. The permselective material may be provided in any of a number of forms, such as those described in U.S. patent application Ser. No. 10/371,148, entitled “Methods and Systems for Controlling and/or Increasing Iontophoretic Flux”, which is incorporated herein by reference.

By way of example without limitation, the permselective material may be provided in a liquid, partially liquid, gelled, partially solid, or fully solid state. In some instances, the permselective material may be supported by a support structure such as an additional membrane having sufficient porosity and chemical inertness so as to avoid interfering with the performance of the permselective material, yet having sufficient mechanical integrity for ease in handling. The material can also be provided in the form of a membrane having a surface sized and/or shaped for direct contact with the eye or shaped for direct contact with the current driving electrode (e.g., Ag/AgCl). In other instances, the permselective material may be comprised of a polyelectrolyte, which can be a single molecule or an aggregate of molecules.

The macrolide can be non-invasively administered into the subject's eye at nearly any location on the eye in accordance with the present invention. However, in one aspect, the macrolide may be delivered to the top of the eye. In another aspect, delivery may be made to the bottom of the eye. In yet another aspect, delivery may be made at, or near the back of the eye. In an additional aspect, delivery may be made to the side of the eye. In a further aspect, delivery may be made simultaneously to different locations of the eye, for example opposite sides of the eye using separate non-invasive delivery devices. It is preferable to deliver the carriers and drugs to a location or locations in the eye that will provide sufficient amount of the drugs to their sites of action for the prevention or treatment of an eye disease. For example, to deliver the macrolide to the anterior and posterior chambers of the eye, the preferred site of iontophoresis application is near the limbus. For the delivery of the macrolide to the back of the eye, the preferred site to deliver the carriers will be in the sclera or in the vitreous. In the sclera, the macrolide will be carried to the back of the eye by the blood vasculature system in the eye. In the vitreous, the macrolide will diffuse to the back of the eye passively.

Additionally, the macrolides formulations according to aspects of the present invention can be administered as controlled release formulations in order to prolong the residence time, and thus the therapeutic effects of the agents in the eye. Further discussion of methods and devices for providing such controlled release can be found in U.S. patent application Ser. Nos. 10/269,911 filed on Oct. 11, 2002, 11/238,144 filed on Sep. 27, 2005, and 11/238,104 filed on Sep. 27, 2005, each of which are incorporated herein by reference in their entirety.

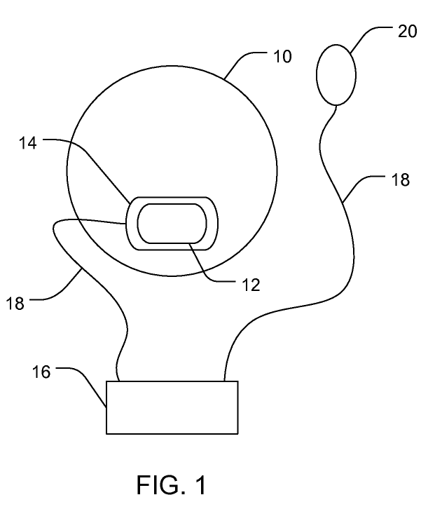

The present invention also encompasses systems and devices for administering macrolides into an eye of a subject. In one aspect, for example, a system for treating or preventing an ocular condition as has been described is provided. As is shown in FIG. 1, such a system may include an ocular iontophoretic device 10 including at least one drug reservoir 12 and a macrolide contained within the drug reservoir. The ocular iontophoretic device 10 further includes an active electrode 14 to provide an electrical current to the drug reservoir 12 and thus iontophoretically drive the macrolide into the eye. The active electrode 14 is electrically coupled to a power supply 16 with an electrical lead 18. The power supply may also function to regulate the electrical current delivered to the active electrode. Additionally, the system may further include a return electrode 20 to complete an electrical circuit. The return electrode 20 may complete the electrical circuit by contacting any surface of the subject's body, including the surface of an eye, an eyelid, a portion of the face or ear, or any other bodily surface that would allow the completion of such a circuit.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

What is claimed is:

1. A method for noninvasively delivering a water insoluble macrolide into an eye of a subject for treatment of an ocular condition, comprising:
administering non-invasively a water soluble form of the macrolide directly into an eye of a subject having or at risk for having the ocular condition, wherein the water soluble form of the macrolide is converted into the water insoluble macrolide in the eye in order to treat the ocular condition.

2. The method of claim 1, wherein the macrolide is a rapamycin agent.

3. The method of claim 1, wherein the macrolide is a member selected from the group consisting of SDZ-RAD, tacrolimus, everolimus, pimecrolimus, CCl-779, AP23841, ABT-578, cyclophilins, FK506-binding proteins (FKBPs), TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, ascomycin, and pharmaceutically acceptable prodrugs, analogs, salts, and esters thereof.

4. The method of claim 2, wherein the rapamycin agent is a water soluble rapamycin agent.

5. The method of claim 2, wherein the rapamycin agent is a prodrug of rapamycin.

6. The method of claim 1, wherein the macrolide is a member selected from the group consisting of ascomycin, and pharmaceutically acceptable prodrugs, analogs, salts, and esters thereof.

7. The method of claim 1, wherein the ocular condition is a member selected from the group consisting of chronic macular edema, diabetic macular edema, cystoid macular edema, macular edema, age related macular degeneration, diabetic retinopathy, urticaria, allergic conjunctivitis, vernal conjunctivitis, inflammation of the eye, allergic responses in the eye, uveitis, Behcet's disease, pars planitis, idiopathic uveitis, ocular sarcoid, sympathetic ophthalmia, idiopathic vitritis, vitritis, uveitis resulting from trauma, iritis, iridocyclitis, scleritis, episcleritis, choroiditis, optic neuritis, Mooren's ulcer, ulcerative keratitis associated with rheumatoid arthritis, anterior uveitis, Thygeson's punctate keratitis, retinitis pigmentosa, diabetic retinopathy, chronic glaucoma, retinal detachment, sickle cell retinopathy, senile macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, retinal vein occlusion, proliferative vitreoretinopathy, angioid streak, retinal artery occlusion, neovascularization due to ocular injury, infections of the eye by infective agents including viruses, fungi, bacteria, and combinations thereof.

8. The method of claim 1, wherein the ocular condition is age related macular degeneration.

9. The method of claim 1, wherein the ocular condition is macular edema.

10. The method of claim 1, further comprising co-administering a vasoconstrictor with the macrolide to enhance the treatment of the ocular condition.

11. The method of claim 10, wherein the vasoconstrictor is an α-agonist.

12. The method of claim 11, wherein the vasoconstrictor is a member selected from the group consisting of naphazoline, tetrahydrozoline, oxymetazoline, and combinations thereof.

13. The method of claim 10, wherein the vasoconstrictor is a sympathomimetic amine.

14. The method of claim 13, wherein the sympathomimetic amine includes a member selected from the group consisting of phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, albuterol, amphetamine, methamphetamine, benzphetamine, ephedrine, phenylpropanolamine, methentermine, phentermine, fenfluramine, propylhexedrine, diethylpropion, phenmetrazine, phendimetrazine, and combinations thereof.

15. The method of either of claim 1, wherein administering is by iontophoretic administration.

16. The method of claim 1, wherein the macrolide is administered as a controlled release formulation.

17. A system for treating an ocular condition as in claim 1, comprising:
an ocular drug delivery device including at least one drug reservoir; and
a macrolide contained within the drug reservoir.

18. The system of claim 17, wherein the macrolide is a member selected from the group consisting of SDZ-RAD, tacrolimus, everolimus, pimecrolimus, CCl-779, AP23841, ABT-578, cyclophilins, FK506-binding proteins (FKBPs), TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, ascomycin, and pharmaceutically acceptable prodrugs, analogs, salts, and esters thereof.

19. The system of claim 17, wherein macrolide is a water soluble rapamycin agent.

20. The system of claim 17, wherein the macrolide is a member selected from the group consisting of ascomycin, and pharmaceutically acceptable prodrugs, analogs, salts, and esters thereof.

21. The system of claim 17, wherein the ocular drug delivery device is an iontophoretic drug delivery device.

22. The system of claim 17, wherein the ocular drug delivery device is a passive drug delivery device.
